(19) United States
(12) Patent Application Publication
(10) Pub. No.: US 2018/0008150 A1
(43) Pub. Date: Jan. 11, 2018

(54) PERSONAL WEARABLE HEADBAND HAVING FAR INFRARED AND TEMPERATURE MEASUREMENT FUNCTIONS

(71) Applicant: HOMEWAY TECHNOLOGY CO., LTD., Tainan City (TW)

(72) Inventors: Tsung-Hsien HSIEH, Tainan City (TW); Tung-Chen HSIEH, Tainan City (TW); Ming-Chia HSIEH, Tainan City (TW)

(21) Appl. No.: 15/412,449

(22) Filed: Jan. 23, 2017

(30) Foreign Application Priority Data
Jul. 5, 2016 (TW) 105210089

(51) Int. Cl. A61B 5/01; A41B 13/00; A61B 5/00; A61N 5/06; A41D 1/00; A41D 20/00

(52) U.S. Cl. CPC A61B 5/01; A41D 1/002; A41D 20/00; A61B 5/6803; A61B 5/7445; A61N 5/0625; A41B 13/00; A61B 5/746; A61B 2503/06; A61B 2560/0214; A61N 2005/0647; A61N 2005/066; A61B 2503/04

(57) ABSTRACT

A personal wearable headband having far infrared and temperature measurement functions includes a headband body defining a headband receiving space and having a plurality of far infrared particles blended in the headband body, a window communicating with the headband receiving space, and a through hole communicating with the headband receiving space and opposite to the window. A temperature measuring unit is disposed in the headband receiving space and includes a temperature measuring element configured to contact the forehead of a user for detecting a temperature thereof, and a display screen corresponding in position to the window and configured to display the temperature detected by the temperature measuring element.

### CROSS-REFERENCE TO RELATED APPLICATION

This application claims priority of Taiwanese Patent Application No. 105210089, filed on Jul. 5, 2016.

### FIELD

The disclosure relates to a headband, and more particularly to a personal wearable headband having far infrared and temperature measurement functions that is suitable to be worn on, for example, the head of an infant or toddler.

### BACKGROUND

During exercise, many people wear headbands about their foreheads for absorbing the sweat and preventing it from entering the eyes or fogging the eyeglasses. Thus, the main function of the conventional headband is to absorb and block the sweat. Some headbands may be printed with beautiful patterns, but they only serve to enhance the aesthetic appearance thereof, and have no other practical uses. When an infant or toddler is sick and has fever, the parent or caregiver must constantly monitor the body temperature change of the infant or toddler. Because the conventional thermometer cannot be worn on a user's body, in circumstances where constant monitoring of the body temperature change of the infant or toddler is required, use thereof is inconvenient.

### SUMMARY

Therefore, an object of the present disclosure is to provide a personal wearable headband having far infrared and temperature measurement functions that can alleviate at least one of the drawbacks of the prior art.

According to this disclosure, a personal wearable headband having far infrared and temperature measurement functions includes a headband body and a temperature measuring unit. The headband body defines a headband receiving space, and has a plurality of far infrared particles blended in the headband body, a window communicating with the headband receiving space, and a through hole communicating with the headband receiving space and opposite to the window. The temperature measuring unit is disposed in the headband receiving space and includes a temperature measuring element configured to contact the forehead of a user for detecting a temperature thereof, and a display screen corresponding in position to the window and configured to display the temperature detected by the temperature measuring element.

The efficiency of this disclosure resides in that by wearing the headband body about the forehead of the user, the temperature of the user's forehead can be easily measured through the temperature measuring unit disposed in the headband body. Further, because the headband body is made of a superfine fibrous fabric having a far infrared function, it can enhance body circulation and metabolism and wearing comfort during use thereof.

### BRIEF DESCRIPTION OF THE DRAWINGS

Other features and advantages of the disclosure will become apparent in the following detailed description of the embodiment with reference to the accompanying drawings, of which:

FIG. 1 is a fragmentary front view of a headband according to the embodiment of this disclosure, illustrating the headband in a non-use position;

FIG. 2 is a fragmentary exploded perspective view of the embodiment;

FIG. 3 is a fragmentary sectional view of the embodiment in an assembled state;

FIG. 4 is a diagram illustrating the components of a temperature measuring unit of the embodiment; and

FIG. 5 illustrates the headband of the embodiment in a use position.

### DETAILED DESCRIPTION

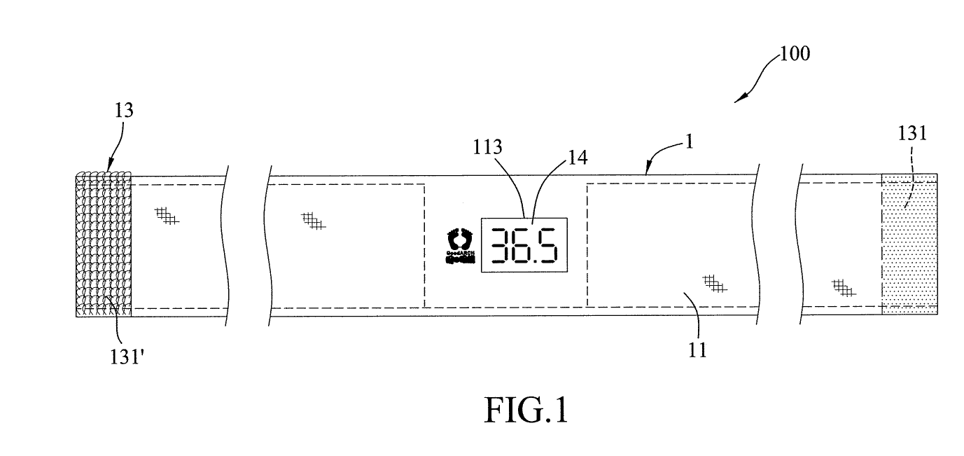

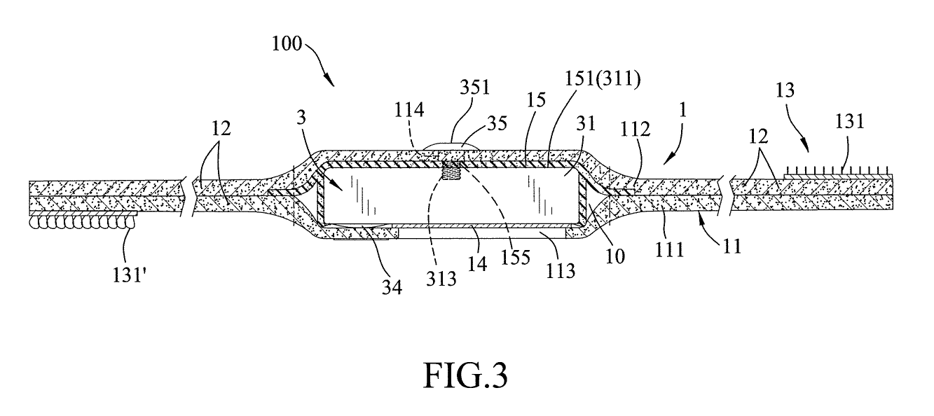

Referring to FIGS. 1 to 5, a headband 100 according to the embodiment of the present disclosure is suitable to be worn about the forehead of a user for measuring the temperature thereof. The headband 100 includes a headband body 1 and a temperature measuring unit 3.

The headband body 1 is elongated, is made of a superfine fibrous fabric having a far infrared function, and includes a headband front wall 111 and a headband rear wall 112 connected to each other by sewing and cooperating with each other to define a headband receiving space 10 therebetween. A plurality of far infrared particles 12 are blended into the superfine fibrous fabric during the manufacturing process thereof. Through this, the far infrared particles 12 do not easily falloff due to friction during use of the headband 100, so that they have a durable effect. Preferably, far infrared powder having the so-called reproductive light with a wavelength ranging from 4˜14 μm is used. In this embodiment, the far infrared powder contains 30% by weight of ZnO₂, 30% by weight of AlO₂, 30% by weight of TiO₂ and 10% by weight of Sb₂O₃. However, it is not limited to the aforesaid disclosure.

The headband receiving space 10 of this embodiment is generally disposed at a central portion of the headband body 1. However, it is not limited thereto. The headband receiving space 10 may be disposed at one side of the headband body 1. Further, there is no restriction on the length of the headband body 1. If the headband 100 is to be used on an infant or toddler, the length of the headband body 1 may be slightly longer than an average head circumference of the infant or toddler. The actual length thereof is about 60 cm. The headband front and rear walls 111, 112 may be integrally formed as one body. In this embodiment, the headband front wall 111 has a window 113 communicating with the headband receiving space 10, and the headband rear wall 112 has a through hole 114 communicating with the headband receiving space 10 opposite to the window 113.

The headband 100 further includes a fastening unit 13, a light transmissive plate 14 and a positioning seat 15. The fastening unit 13 is configured to releasably fasten the headband body 1 on the user's forehead, and includes a hook fastener strip 131 and a loop fastener strip 131′ respectively fixed on two ends of the headband body 1 which are opposite to each other along a length direction thereof and detachably engageable with each other. Preferably, the strips 131, 131′ are made of a soft material suitable for the infant and toddler. Further, there is no restriction on the form of the fastening unit 13. Apart from the disclosed hook and loop fastener strips 131, 131′, the fastening unit 13 may include snap fasteners, releasable adhesive tapes, or a garter. As long as the fastening unit may make the headband body 1 to form a ring, any fastening unit is acceptable.

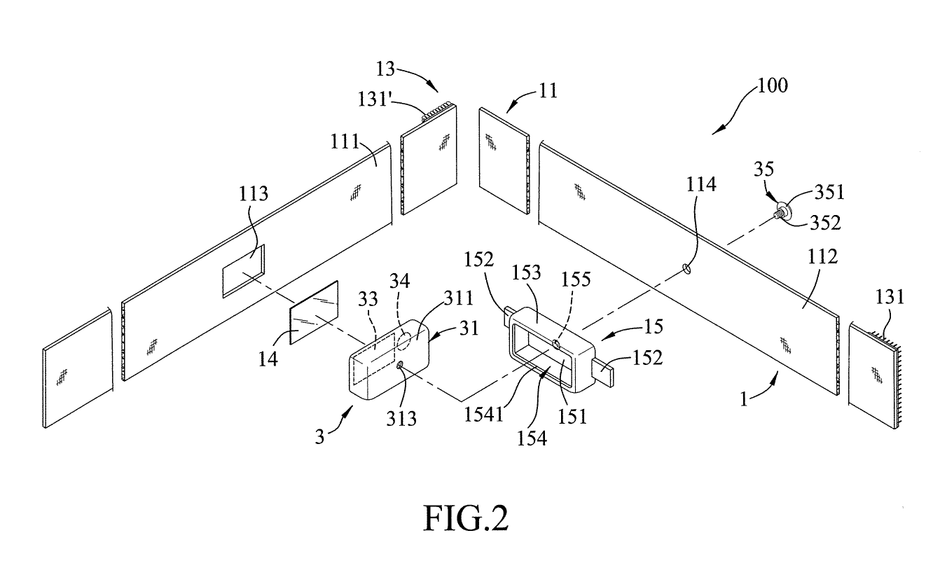

The light transmissive plate 14 is attached to a rear side of the headband front wall 111 within the headband receiving space 10, and corresponds in position to the window 113. The positioning seat 15 is disposed in the headband receiving space 10, is a soft case made of, for example, a silicone material, and includes a base wall 151 abutting against a front side of the headband rear wall 112, a surrounding wall 153 extending forwardly and transversely from an end periphery of the base wall 151 and cooperating with the same to define an accommodation space 154 having an opening 1541 that faces forward, and two wing walls 152 protruding outwardly and oppositely from the base wall 151. The base wall 151 has an aperture 155 corresponding in position to and located in front of the through hole 114. Each wing wall 152 is fixed between the headband front and rear walls 111, 112, as shown in FIG. 3.

The temperature measuring unit 3 of this embodiment includes a housing 31, a circuit board 32 disposed in the housing 31, and a display screen 33, a switch 34, a temperature measuring element 35 and a buzzer 36 electrically connected to the circuit board 32. The housing 31 may be made of a hard plastic material or a soft material, such as silicone. Preferably, the housing 31 is made of a soft material. The circuit board 32 may also be a soft circuit board.

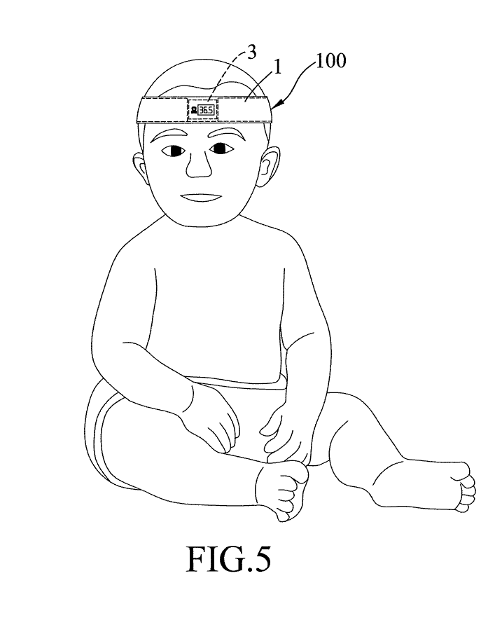

With reference to FIGS. 2 to 4, the housing 31 is removably received in the accommodation space 154 via the opening 1541, and includes a housing rear wall 311 abutting against the base wall 151 and having an engaging hole 313 correspondingly aligned with the aperture 155. In this embodiment, the engaging hole 313 is a threaded hole. The display screen 33 is disposed on a front side of the housing 31 opposite to the housing rear wall 311, and corresponds in position to the light transmissive plate 14. The temperature measuring element 35 includes a threaded stem 352 extending through the through hole 114 and the aperture 155 and threadedly engaging the engaging hole 313, and an arcuate temperature sensing head 351 connected to a rear end of the threaded stem 352 and located externally of the headband rear wall 112. Because the temperature measuring element 35 is threadedly connected to the housing 31, the position thereof can be adjusted in a front-rear direction according to the requirement so as to accommodate different use situations. The switch 34 is disposed on the front side of the housing 31 proximate to the display screen 33, and faces the headband front wall 111. If necessary, a mark or design may be provided on a front side of the headband front wall 111 that corresponds in position to the switch 34 so as to facilitate an operation thereof. In this embodiment, the temperature measuring element 35 and the display screen 33 are electrically connected to each other through the circuit board 32. Alternatively, the temperature measuring element 35 and the display screen 33 may be connected directly and electrically to each other through a wire.

With reference to FIGS. 1, 3 and 5, because the positioning seat 15 is disposed in the headband receiving space 10 and the housing 31 of the temperature measuring unit 3 is disposed in the accommodation space 154 of the positioning seat 15, the housing 31 can be stably positioned in the headband receiving space 10 through the positioning seat 15. After the housing 31 is positioned in the positioning seat 15, the display screen 33 corresponds in position to the window 113, and is located rearwardly of the light transmissive plate 14. The light transmissive plate 14 is disposed between the window 113 and the display screen 33 to protect the latter. Further, the temperature sensing head 351 is located externally of the headband rear wall 112.

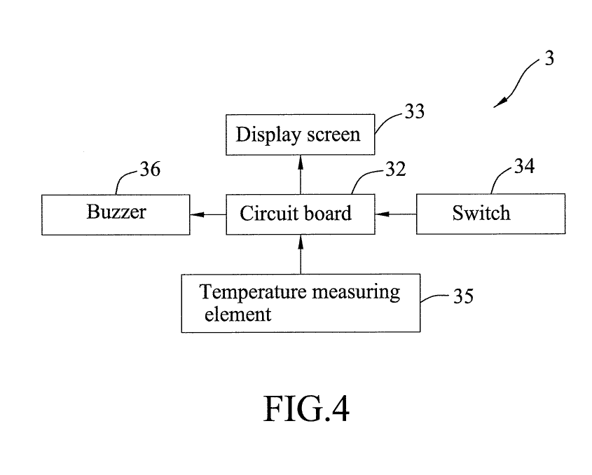

When the headband 100 is in a non-use position, as shown in FIGS. 1 and 3, the hook and loop fastening strips 131, 131′ are opposite to each other along a length direction of the headband body 1. To wear the headband 100 on the user's forehead, the side thereof having the temperature sensing head 351 is first placed on the user's forehead such that the temperature sensing head 351 contacts the user's forehead and the display screen 33 faces forwardly, after which the two opposite ends of the headband body 1 are pulled rearward and the hook and loop fastening strips 131, 131′ are engaged to each other so as to place the headband 100 in a use position, as shown in FIG. 5. After the headband 100 is positioned about the user's forehead, a parent or a caregiver may press the switch 34 to operate the temperature measuring unit 3. The temperature of the user detected by the temperature measuring element 35 is then displayed on the display screen 33. Hence, the temperature measurement function of the wearable headband 100 can be achieved. Further, to remove and replace a battery unit in the housing 31, the headband body 1 may be provided with a mouth (not shown) located on a top side thereof and communicating with the headband receiving space 10 to facilitate removal and mounting of the housing 31.

With reference to FIGS. 3 to 5, it is worth to mention herein that, because the headband body 1 is made of a superfine fibrous fabric having a far infrared function, when the headband 100 is worn, apart from being able to measure the body temperature of the user, the far infrared particles 12 thereof can generate wavelengths beneficial to the user's body. Hence, the headband 100 of this disclosure can enhance body circulation and metabolism and wearing comfort during use thereof. Further, when the detected body temperature is higher than 38° C., the buzzer 36 produces an intermittent alarm sound; when the detected body temperature is higher than 39° C., the buzzer 36 produces an intermittent and short alarm sound; and when the detected body temperature is higher than 40° C., the buzzer 36 produces a continuous alarm sound. Thus, the buzzer 36 of this embodiment can produce different alarming signals according to the detected varying degrees of fever. The headband 100 of this disclosure is especially suitable to be worn on the head of an infant or toddler who cannot express their sickness in words.

In sum, the headband 100 of this disclosure not only can detect the body temperature of the user, but also is comfortable to wear and can enhance body circulation and metabolism. Since the headband 100 can be stably worn for a long time, it is very suitable for use in detecting the body temperature change of an infant or toddler. Further, the buzzer 36 provided on the headband 100 can be used to convert the detected temperature into alarm sounds, so that the headband 100 is also provided with a warning effect. Hence, the headband 100 of this disclosure has a novel structure, and is convenient and practical to use.

While the disclosure has been described in connection with what is considered the exemplary embodiment, it is understood that this disclosure is not limited to the disclosed embodiment but is intended to cover various arrangements included within the spirit and scope of the broadest interpretation so as to encompass all such modifications and equivalent arrangements.

What is claimed is:

1. A personal wearable headband having far infrared and temperature measurement functions, comprising:
a headband body defining a headband receiving space and having a plurality of far infrared particles blended in said headband body, a window communicating with said headband receiving space, and a through hole communicating with said headband receiving space and opposite to said window; and
a temperature measuring unit disposed in said headband receiving space and including a temperature measuring element configured to contact the forehead of a user for detecting a temperature thereof, and a display screen corresponding in position to said window and configured to display the temperature detected by said temperature measuring element.

2. The personal wearable headband as claimed in claim 1, wherein said headband body includes a headband front wall having said window, and a headband rear wall having said through hole, said headband front and rear walls being connected to and cooperating with each other to define said headband receiving space.

3. The personal wearable headband as claimed in claim 2, further comprising a fastening unit configured to releasably fasten said headband body on the user's forehead, said fastening unit including a hook fastener strip and a loop fastener strip respectively fixed on two ends of said headband body which are opposite to each other along a length direction thereof and releasably engageable with each other.

4. The personal wearable headband as claimed in claim 2, wherein said temperature measuring unit further includes a housing, a circuit board disposed in said housing and connected electrically to said display screen and said temperature measuring element, and a switch connected electrically to said circuit board and facing said headband front wall.

5. The personal wearable headband as claimed in claim 4, further comprising a positioning seat disposed in said headband receiving space, said housing being positioned in said positioning seat.

6. The personal wearable headband as claimed in claim 5, wherein said positioning seat includes a base wall abutting against a front side of said headband rear wall, a surrounding wall extending forwardly and transversely from an end periphery of said base wall and cooperating with the same to define an accommodation space, and two wing walls protruding outwardly and oppositely from said base wall, each of said wing walls being fixed between said headband front and rear walls, said base wall having an aperture corresponding in position to and located in front of said through hole, said housing being removably received in said accommodation space.

7. The personal wearable headband as claimed in claim 4, wherein said housing includes a housing rear wall abutting against said base wall and having an engaging hole correspondingly aligned with said aperture, said temperature measuring element being adjustably connected to said housing in a front-rear direction and including a threaded stem extending through said through hole and said aperture and threadedly engaging said engaging hole, and a temperature sensing head connected to a rear end of said threaded stem and located externally of said headband rear wall for contacting the forehead of the user.

8. The personal wearable headband as claimed in claim 4, further comprising a light transmissive plate disposed between said window and said display screen.

9. The personal wearable headband as claimed in claim 4, wherein said temperature measuring unit further includes a buzzer connected electrically to said circuit board.
